(19) United States
(12) Patent Application Publication
(10) Pub. No.: US 2026/0224327 A1
(43) Pub. Date: Aug. 6, 2026

(54) MONITORING UTILIZATION OF REUSABLE SURGICAL DEVICES

(71) Applicant: Alcon Inc., Fribourg (CH)

(72) Inventor: Kevin Canzone, Coto de Caza, CA (US)

(21) Appl. No.: 19/449,631

(22) Filed: Jan. 15, 2026

Related U.S. Application Data
(60) Provisional application No. 63/754,723, filed on Feb. 6, 2025.

(51) Int. Cl. A61B 90/98; A61B 50/33; A61B 90/00

(52) U.S. Cl. CPC A61B 90/98; A61B 50/33; A61B 2090/0805

(57) ABSTRACT

Certain aspects of the present disclosure provide systems and methods for monitoring usage of reusable surgical devices. In certain embodiments, an apparatus includes a housing defining an external reader surface, and a wireless reader comprising an antenna disposed within the housing. Within a bandwidth of the antenna, a majority of a radiation pattern of the antenna extends through the external reader surface. The apparatus further includes a reflector disposed near the antenna opposite the external reader surface. The reflector is configured to reorient a portion of the radiation pattern as a reflected portion that extends through the external reader surface.

### INTRODUCTION

Many surgical devices are designed to be safely reused in multiple surgical procedures, although the ability to reuse surgical devices can be limited. Reusable surgical devices can be employed in different surgical environments and with different surgical systems, which can make the utilization of the surgical devices difficult to track. Transponders (or “tags”) in the reusable surgical devices may be used to track the usage thereof. The compact form factor of many reusable surgical devices often dictates a relatively small size for an antenna connected to the transponder, making it difficult to couple sufficient electrical energy into the transponder to supply power to, and/or to communicate with, the electronics of the transponder.

### SUMMARY

The present disclosure relates to surgical systems and methods, and more particularly, to systems and methods for monitoring utilization of reusable surgical devices.

In certain embodiments, one general aspect includes an apparatus to monitor usage of a reusable surgical device. The apparatus includes a housing defining an external reader surface, and a wireless reader comprising an antenna disposed within the housing. Within a bandwidth of the antenna, a majority of a radiation pattern of the antenna extends through the external reader surface. The apparatus further includes a reflector disposed near the antenna opposite the external reader surface. The reflector is configured to reorient a portion of the radiation pattern as a reflected portion that extends through the external reader surface.

In certain embodiments, another general aspect includes a method to monitor usage of a reusable surgical device. The method includes transmitting, using an antenna of a wireless reader, an interrogation signal to a transponder of the reusable surgical device disposed near an external reader surface. A portion of a radiation pattern of the antenna is reoriented through the external reader surface by a reflector that is disposed near the antenna opposite the external reader surface. The method further includes receiving, at the wireless reader, a response from the transponder. The method further includes determining, by a surgical console connected with the wireless reader, an amount of previous usage of the reusable surgical device that is based at least partly on the response. The method further includes transmitting, by the surgical console, a first control signal that allows or denies usage of the reusable surgical device in a surgical procedure, the first control signal based at least partly on the amount of previous usage.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope and may admit to other equally effective embodiments.

FIG. 1 illustrates an exemplary surgical environment according to certain embodiments of the present disclosure.

FIG. 2 illustrates an exemplary connection of a reusable surgical device with a wireless reader according to certain embodiments of the present disclosure.

FIG. 3 is a block diagram of an exemplary surgical system according to certain embodiments of the present disclosure.

FIG. 4 illustrates an exemplary radiation pattern of an antenna according to certain embodiments of the present disclosure.

FIG. 5 illustrates an exemplary apparatus having a reflector to reorient a portion of the radiation pattern of the antenna according to certain embodiments of the present disclosure.

FIG. 6 illustrates a method to monitor usage of a reusable surgical device according to certain embodiments of the present disclosure.

To facilitate understanding, identical reference numerals have been used, where possible, to designate identical elements that are common to the Figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

The number and complexity of surgical procedures, including ophthalmic procedures such as vitreoretinal surgery, continues to increase every day. Various surgical devices, including surgical tools such as ophthalmic handpieces and their components, may be utilized to perform the surgical procedures. Although some surgical devices can be sterilized and safely reused, the surgical devices have a finite lifetime and should occasionally be discarded and/or replaced. Therefore, it can be advantageous to monitor parameters related to utilization of the surgical devices, such as a shelf life, a usage amount, and so forth.

Reusable surgical devices can be employed in different surgical environments and with different surgical systems, making the utilization of the reusable surgical devices difficult to track. Contact-based tracking methods are generally unsuitable for sterile environments due to risk of contamination. Further, adding labels or marks (such as a barcode or quick response (QR) code) to packaging may not be sufficient to track utilization locally at the reusable surgical devices, as the labels or marks are generally not able to be updated with utilization information.

The present disclosure describes examples of monitoring the utilization of reusable surgical devices, for example, by maintaining device-specific profile data in memory embedded in, coupled to, or otherwise natively associated with the reusable surgical devices. In some embodiments, the memory resides in radio frequency identification (RFID) or near-field communication (NFC) transponders (or “tags”), for example, so as to enable non-contact access. In various embodiments, an electronic device associated with a surgical environment, such as a surgical console or a component thereof, can assess an acceptability of a given surgical device at or near the time of a surgical procedure, for example, based on device-specific profile data retrieved from the memory associated with the reusable surgical device. In various embodiments, the electronic device can deny usage of the reusable surgical device in the surgical procedure when it determines that the device is unacceptable, for example, due to diminished safety, utility, and/or the like. In various embodiments, the electronic device may further update the device-specific data to include new information, such as data reflective of the reusable surgical device’s usage in the surgical procedure at issue. Accordingly, surgical use of the reusable surgical device can be tracked and the reusable surgical device can be discarded and/or replaced when its usage limit is reached.

Advantageously, in certain embodiments, systems described herein can monitor utilization of reusable surgical devices without any external storage of device-specific profile data, such as in network, system, or cloud storage locations. In certain embodiments, since the device-specific profile data may be stored and maintained in the memory natively associated with the reusable surgical devices, such data travels with the devices as they are used within different environments and systems. Further, in embodiments in which RFID, NFC, or similar protocols are utilized for data transfer, the device-specific data can be retrieved and updated without contamination risk to the reusable surgical devices, thus rendering the method suitable for sterile environments such as those involving sterile surgical ophthalmic devices.

The compact form factor of many reusable surgical devices often dictates a relatively small size for an antenna connected to the transponder, making it difficult to couple sufficient electrical energy into the transponder to supply power to, and/or to communicate with, the electronics of the transponder. In some embodiments, an apparatus includes a reflector (such as a parabolic reflector) that is disposed near the antenna opposite an external reader surface, and the reflector reorients a portion of a radiation pattern of the antenna as a reflected portion that extends through the external reader surface. For example, the antenna may be tuned such that, within a bandwidth of the antenna, a majority of the radiation pattern (e.g., a main lobe and optionally one or more side lobes) extends through the external reader surface, and some of the radiation pattern (e.g., a back lobe and optionally one or more side lobes) extends in a direction away from the external reader surface. At least a portion of the energy that radiates away from the external reader surface is redirected by the reflector, such that the redirected energy also extends through the external reader surface.

With the additional redirected energy from the radiation pattern, the apparatus provides improved coupling with the antenna of the transponder, which may support orientations of the surgical device that are further from those orientations providing maximum coupling (e.g., parallel antenna planes of the wireless reader and the reusable surgical device). For example, the antenna plane of the reusable surgical device may be orthogonal to the antenna plane of the wireless reader. Such orientations may lead to more efficient and/or more accurate surgical procedures, as those surgeons or technicians handling the reusable surgical device may scan the reusable surgical device at the wireless reader with reduced effort. For example, the scan may be completed using a natural carrying motion of the reusable surgical device to, or past, the wireless reader, may have a greater likelihood of being completed successfully on a first attempt, and so forth.

The improved coupling with the antenna of the transponder may enable other features. In one example, the improved coupling may support more compact implementations of the wireless reader and/or the reusable surgical device. In another example, the improved coupling may support reduced power consumption by the wireless reader.

In yet another example, the transponder may operate at frequencies outside of the bandwidth of the antenna (of the reusable surgical device). Generally, the radiation pattern of an antenna varies by frequency, such that the various lobes of the radiation pattern have different shapes and/or orientations at different frequencies. Assuming the transponder includes an antenna tuned for operation in a first location (e.g., Europe, between 860-870 megahertz (MHz)) while the antenna of the reusable surgical device is tuned for operation in a second location (e.g., the U.S., between 902-928 MHz), the improved coupling provided by the apparatus can enable interoperability between the reusable surgical device and the wireless reader.

As used herein, the term “surgical system” may refer to any surgical system, console, or device for performing a surgical procedure. For example, the term “surgical system” may refer to a surgical console, such as a phacoemulsification console, a vitrectomy console, a laser system, or any other consoles, systems, or devices used in an ophthalmic operating room, as known to one of ordinary skill in the art. Note that although certain embodiments herein are described in relation to ophthalmic systems, devices, and environments, the embodiments described herein are similarly applicable to other types of medical or surgical systems, devices, and environments.

As used herein, the term “sensor” may refer to any type of device that detects or measures, e.g., a physical input, and records, indicates, or otherwise responds to the physical input. For example, the term “sensor” may refer to a device configured to detect or measure a position, location, proximity (e.g., to a surgical console), tilt, height, speed (e.g., an accelerometer), temperature, etc., of a surgical device, system, or user. In certain examples, the term “sensor” may refer to a device configured to detect touch, i.e., touching of a surgical device or system by a user, such as a capacitive- or resistive-type touch sensor. In certain examples, the term “sensor” may refer to an imaging device configured to detect and relay image-based information, such as a charge-coupled device (CCD) or an active-pixel sensor (APS), such as a complementary metal-oxide-semiconductor (CMOS) sensor.

Although generally described with reference to ophthalmic surgical devices and systems, the devices and systems described herein may be implemented with other devices and systems, such as devices and systems for other surgeries, without departing from the scope of the present application.

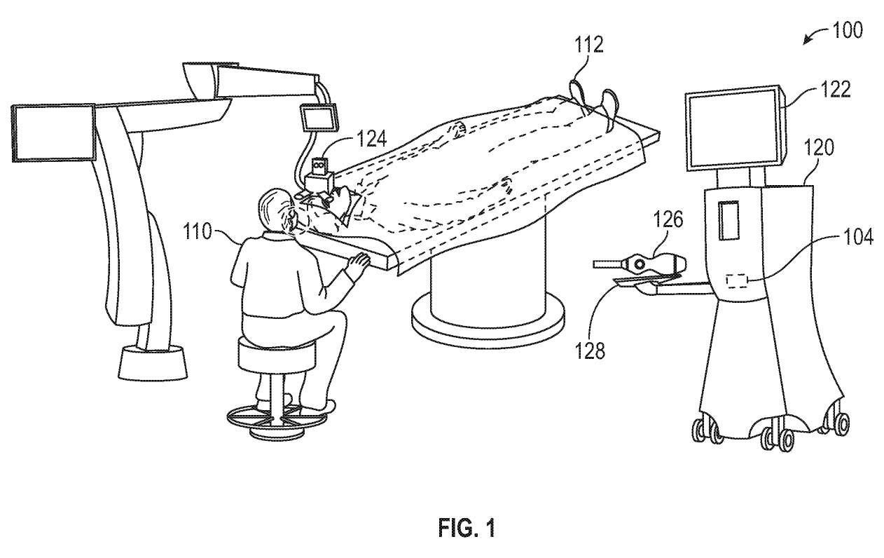

FIG. 1 illustrates an exemplary surgical environment 100, such as an ophthalmic operating environment, in which a surgical console 120 may be utilized for performance of a surgical procedure, according to embodiments of the present disclosure. As shown, the surgical environment 100 further includes a surgeon 110, a patient 112, as well as a plurality of surgical systems and devices, such as the surgical console 120 having a display device 122, a microscope system 124, a surgical tool 126, and a surgical tray 128. Generally, examples of suitable surgical systems that may be included in the surgical environment 100 include surgical devices and consoles for performing vitreoretinal procedures, cataract surgeries, corneal transplants, glaucoma surgeries, LASIK (Laser-Assisted In Situ Keratomileusis) surgeries, refractive lens exchanges, trabeculectomies, keratotomy procedures, and keratoplasty surgeries, or other devices and consoles identifiable by those of ordinary skill. Consoles that are capable of performing two or more of these procedures are also within the scope of this disclosure. An example of a console configured for performing vitreoretinal procedures is the Constellation® System available from Alcon Laboratories, Inc., Fort Worth, Texas. An example of a console configured for performing cataract surgeries is the Centurion® System available from Alcon Laboratories, Inc., Fort Worth, Texas.

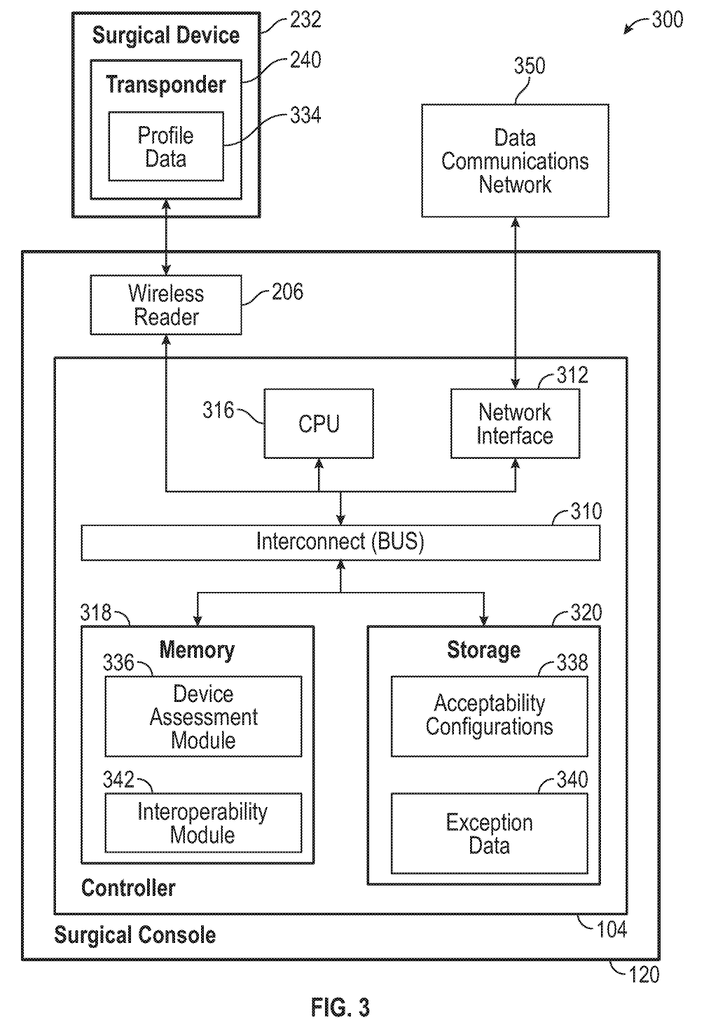

The surgical console 120 includes a controller 104 representing one example implementation of an electronic device, discussed in greater detail below with respect to FIG. 3. As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

The electronic device comprises one or more processors and a memory. The one or more processors are any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some aspects, the electronic circuitry is configured to perform any of the functions described herein. Further, the one or more processors are not limited to a single processing device and may encompass multiple processing devices.

The one or more processors may include other hardware that operates software to control and process information. In some aspects, the one or more processors execute software stored in the memory to perform any of the functions described herein. The one or more processors control the operation and administration of the electronic device by processing information (e.g., information received from input devices and/or communicatively coupled electronic devices).

The memory may store, either permanently or temporarily, data, operational software, or other information for the one or more processors. The memory may include any one or a combination of volatile or non-volatile local or remote devices suitable for storing information. For example, the memory may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory, a disk, a CD (compact disc), or a flash drive. In particular embodiments, the software may include an application executable by the one or more processors to perform one or more of the functions described herein.

The controller 104 is configured to cause the surgical console 120 to perform one or more tasks for driving a surgical tool, e.g., the surgical tool 126. The surgical tool 126 can be any type of tool (e.g., probe, hand-piece, etc.) used during an operation. For example, the surgical tool 126 can be a tool used during a surgical procedure, such as an ophthalmic surgical procedure. Examples of surgical tool 126 can include a cataract handpiece, a vitrectomy handpiece, a phacoemulsification handpiece, and so forth. Further, the techniques discussed herein may be used with any type of surgical tool for which tracking of the usage of the surgical tool may be beneficial.

In the example of FIG. 1, the controller 104 is integrated within the surgical console 120. In certain other embodiments, the controller 104 is a stand-alone device or module that is in wireless or wired communication with, e.g., the surgical console 120 and other devices within the surgical environment 100. In certain aspects, operations of the controller 104 may be executed partly by the one or more processors associated with the controller 104 and/or the surgical console 120, and partly by one or more remote processors (e.g., of one or more networked electronic devices, such as in a public or private cloud).

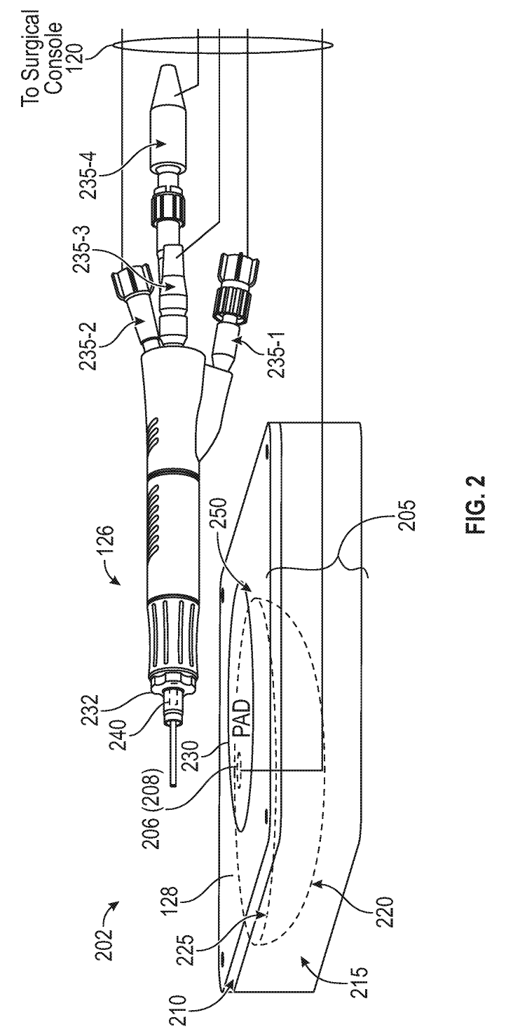

FIG. 2 illustrates an exemplary connection of a reusable surgical device with a wireless reader according to certain embodiments of the present disclosure. The features illustrated in FIG. 2 may be used in conjunction with other embodiments. For example, FIG. 2 may represent a portion of the surgical environment 100 of FIG. 1 in greater detail. In the example of FIG. 2, the surgical tool 126 is an ophthalmic handpiece having a reusable surgical device 232 as an attachment thereto. The surgical tool 126 further comprises a plurality of ports 235-1, 235-2, 235-3, 235-4 that support the functionality of the surgical tool 126 and that are in communication with the surgical console 120, e.g., by tubing, cabling, optical fibers, etc. For example, the plurality of ports 235-1, 235-2, 235-3, 235-4 may be operated to supply and/or remove fluids, to supply electrical power to the surgical tool 126, to communicate electrical and/or optical signals between the surgical tool 126 and the surgical console 120, and so forth. The reusable surgical device 232 is shown to be a removable component of the surgical tool 126 that, in a typical embodiment, is reusable in multiple surgical procedures in accordance with medical standards or practices.

FIG. 2 depicts an apparatus 202 that is configured to monitor usage of the reusable surgical device 232. The apparatus 202 comprises a housing 205, which as shown is formed of an upper housing 210 and a lower housing 215 that are connected or attached together using any suitable techniques. Various components of the apparatus 202 are disposed within an internal volume defined by the housing 205, as will be discussed in greater detail below. Other implementations of the housing 205 may be formed as a single member, e.g., formed around the various components.

The housing 205 may be integrated into one or more components of the surgical environment 100. In some embodiments, the housing 205 is integrated into the surgical tray 128. The surgical tray 128 is shown to have a surgical pad 230 in proximity thereto (e.g., at or near a top surface of the surgical tray 128), such that the surgical tool 126 can be placed on the surgical pad 230 in preparation for the surgical procedure. It should be appreciated that the surgical pad 230 can be any suitable size, shape, or layout relative to the surgical tray 128. In some embodiments, the surgical pad 230 is not discretely implemented but various components of the surgical pad 230 may be implemented in other equipment, such as directly in the surgical tray 128, and/or the like.

In certain embodiments, the surgical pad 230, the surgical tray 128, and/or the surgical console 120 can include a usage sensor that is configured to detect placement of the surgical tool 126 on the surgical pad 230. For example, the usage sensor can be a laser sensor, weight sensor, proximity sensor, or other type of similar device that is positioned and configured to detect placement of the surgical tool 126 on or near the surgical pad 230, which can provide an indication to the controller 104 of an intent to use the surgical tool 126 having the reusable surgical device 232 connected thereto.

The housing 205 includes, or is associated with, a wireless reader 206 that is in communication with the controller 104, while the reusable surgical device 232 includes, or is associated with, a transponder 240. The wireless reader 206 can include any suitable interface for non-contact, wireless communication (e.g., one-way or two-way signals) between the controller 104 and, e.g., the transponder 240. For example, the wireless reader 206 (sometimes referred to as an “interrogator”) may be, or include, an RFID reader, an NFC reader, or another wireless-type reader or receiver using any suitable protocols. In similar fashion, the transponder 240 may be, or include, a passive or active RFID or NFC transponder, sometimes referred to as a “tag”, or another similar device for transmitting (i.e., communicating) device-specific profile data to the wireless reader 206. In some cases, the wireless reader 206 and the transponder 240 may be configured for ultra high frequency (UHF) RFID communication. For illustrative purposes, examples may be periodically described herein in which the wireless reader 206 and the transponder 240 are configured for RFID communication.

In some embodiments, the wireless reader 206 comprises an antenna 208 disposed within the housing 205. The antenna 208 may have any implementation suitable for RFID communication, such as a planar disk antenna, a bent dipole antenna, a coupled antenna, a single-ring monopole antenna, and so forth. Further, the antenna 208 may be formed of one antenna element or multiple antenna elements. In some embodiments, the antenna 208 is disposed substantially within a plane, and the radiation pattern of the antenna 208 comprises one or more lobes projecting outward from the plane. In some embodiments, the antenna 208 is arranged within the housing 205, and is tuned such that, within a bandwidth of the antenna 208, a majority of the radiation pattern of the antenna 208 extends through an external reader surface 250 of the housing 205. In some embodiments, the one or more lobes of the radiation pattern comprise a main lobe having the greatest power or field strength of the one or more lobes, and the main lobe and optionally one or more side lobes extends through the external reader surface 250 of the housing 205.

In some embodiments, the plane of the antenna 208 is parallel to the external reader surface 250. In some embodiments, the external reader surface 250 corresponds to a top surface of the housing (or in some cases, of the surgical pad 230 and/or the surgical tray 128). The antenna 208 (and the wireless reader 206) may have any suitable disposition relative to the housing 205, the surgical tray 128, and/or the surgical pad 230. In some embodiments, the antenna 208 is coaxially aligned with the surgical pad 230. In other embodiments, a center axis of the antenna 208 is offset from a center axis of the surgical pad 230. However, other orientations and/or configurations of the housing 205, the external reader surface 250, and the antenna 208 are also contemplated. For example, the wireless reader 206 may be mounted such that the plane of the antenna 208 has a vertical orientation and the external reader surface 250 is a side surface of the housing 205.

The apparatus 202 further comprises a reflector 220 that is disposed near the antenna 208 opposite the external reader surface 250. The reflector 220 is configured to reorient a portion of the radiation pattern as a reflected portion that extends through the external reader surface 250. For example, a back lobe and optionally one or more side lobes may extend away from the external reader surface 250, and the reflector 220 redirects some or all of this energy toward the external reader surface 250, improving the coupling of the wireless reader 206 with the transponder 240.

The reflector 220 may have any suitable implementation, such as a substantially continuous surface, a surface having one or more discontinuities, multiple surfaces or reflector elements, and so forth. The reflector 220 may be formed of a metal such as copper, aluminum, or iron, and may be formed as a solid shape or as a sheet. In some embodiments, the reflector 220 comprises a parabolic reflector having a wide end 225 (e.g., opposite a vertex of the parabolic reflector) near the antenna 208. The parabolic reflector is configured to converge at least some of the reflected portion of the radiation pattern toward a focus, which in some cases can be disposed above the external reader surface 250. Other types of surfaces and curvatures of the reflector 220 are also contemplated. For example, the reflector 220 may have a conical surface, a substantially parabolic surface formed of a plurality of flat surface elements, an asymmetric surface, and so forth. Some other examples of the reflector 220 include a passive element configured to absorb and directionally re-radiate energy from the antenna, a flat reflector, a corner reflector, a cylindrical reflector, and so forth. In some embodiments, the dimensions of the reflector 220 and/or the spacing between the reflector 220 and the antenna 208 is based on a beam angle of the antenna 208 and a distance corresponding to the focus of the reflector 220 or an expected location of the reusable surgical device 232 during reading. In some embodiments, the dimensions and the spacing may be determined using optimization software.

The reflector 220 may be disposed within the housing 205 or external thereto (e.g., connected to a bottom surface of the housing 205). In some embodiments, the reflector 220 is coaxially aligned with the antenna 208.

In some embodiments, the transponder 240 includes a memory that stores profile data about the reusable surgical device 232. The profile data can include various device-specific parameters such as, for example, a device identifier, a usage value indicating an amount of usage of the reusable surgical device 232, a manufacturing date, a manufacturing lot identifier, a manufactured power output, and/or other information. In various embodiments, the profile data stored by the transponder 240 can be updated by the controller 104 via the wireless reader 206. One skilled in the art will appreciate that the transponder 240 can be associated with the reusable surgical device 232 in any suitable fashion, including being embedded, positioned, and/or mounted in or on any suitable location relative to the reusable surgical device 232. In the illustrated embodiment, the transponder 240 is shown to be embedded within the reusable surgical device 232.

During performance of an ophthalmic surgical procedure on the patient 112, the surgeon 110 may utilize one or more surgical devices, including the surgical tool 126 having the reusable surgical device 232 as described previously. In certain embodiments, the controller 104 is operable to monitor utilization of the reusable surgical device 232, as well as the utilization of other similarly configured surgical devices, using the profile data stored in the transponder 240. For example, in certain embodiments, prior to the performance of the ophthalmic surgical procedure, the reusable surgical device 232 may be read by the wireless reader 206 when resting on the surgical pad 230 or when moved over or past the external reader surface 250 by the surgeon 110. Responsive to identifying the reusable surgical device 232, the controller 104 can automatically determine the acceptability (e.g., safety and/or utility) of the reusable surgical device 232 for the ophthalmic surgical procedure and may deny usage of the reusable surgical device 232 if the reusable surgical device 232 is deemed unacceptable. In one example, the controller 104 may deny usage by transmitting a signal that causes the transponder 240 to write a predetermined value to the memory indicating the reusable surgical device 232 should no longer be used. In another example, the controller 104 may alter or disable communication from the surgical console 120 to one or more of the plurality of ports 235-1, 235-2, 235-3, 235-4 to inhibit nominal operation of the surgical tool 126. Further, in various embodiments, the controller 104 is operable to update the profile data stored in the transponder 240 to reflect additional usage of the reusable surgical device 232.

FIG. 3 is a block diagram 300 of an exemplary surgical system according to certain embodiments of the present disclosure. The features illustrated in the block diagram 300 may be used in conjunction with other embodiments. For example, the block diagram 300 may represent the communication and interoperation of the components of the surgical environment 100 of FIG. 1.

As shown, the transponder 240 associated with the reusable surgical device 232 includes profile data 334 in its memory. The profile data 334 can include any parameter or combination of parameters descriptive of the reusable surgical device 232 such as identification information (e.g., a unique identifier, a manufacturing date, a manufacturing lot identifier, a manufactured power output) and usage data (e.g., a usage value indicating an amount of previous usage of the reusable surgical device 232).

In various embodiments, at least a portion of the profile data 334 is updatable. For example, the usage value mentioned above can be adjusted or updated in response to further usage of the reusable surgical device 232. For example, if the usage value is reflective of a number of uses, the usage value can be an integer that begins at zero and is incremented with each additional surgical use of the reusable surgical device 232. Alternatively, in cases where the usage value is reflective of a number of uses, the usage value can be an integer that begins at predetermined upper boundary (e.g., ten) and is decremented with each additional surgical use of the reusable surgical device 232. In other examples, the usage value can be reflective of other indicators of use, such as time in use, duty cycle, operational power level, etc., such that the usage value is either increased or decreased, as appropriate, responsive to each additional surgical use.

As shown, the surgical console 120 includes, without limitation, the controller 104 and the wireless reader 206, which enable connection of the controller 104 to the transponder 240 of the reusable surgical device 232. As mentioned above, the controller 104 represents one example implementation of an electronic device. As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. As shown, the controller 104 includes a network interface 312 for connection with data communications network 350.

An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

The electronic device comprises one or more processors and a memory. The one or more processors are any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some aspects, the electronic circuitry is configured to perform any of the functions described herein. Further, the one or more processors are not limited to a single processing device and may encompass multiple processing devices. As shown, the controller 104 includes a central processing unit (CPU) 316.

The one or more processors may include other hardware that operates software to control and process information. In some aspects, the one or more processors execute software stored in the memory to perform any of the functions described herein. The one or more processors control the operation and administration of the electronic device by processing information (e.g., information received from input devices and/or communicatively coupled electronic devices).

The memory may store, either permanently or temporarily, data, operational software, or other information for the one or more processors. The memory may include any one or a combination of volatile or non-volatile local or remote devices suitable for storing information. For example, the memory may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory, a disk, a CD, or a flash drive. In particular embodiments, the software may include an application executable by the one or more processors to perform one or more of the functions described herein. As shown, the controller 104 includes a memory 318 and storage 320.

The CPU 316 may retrieve and store application data in the memory 318, as well as retrieve and execute instructions stored in the memory 318. An interconnect 310 transmits programming instructions and application data among the CPU 316, the network interface 312, the memory 318, the storage 320, and the reusable surgical device 232. The CPU 316 can represent a single CPU, multiple CPUs, a single CPU having multiple processing cores, and the like. The memory 318 represents volatile memory of the controller 104 such as random access memory. The storage 320 represents non-volatile memory of the controller 104 such as a disk drive. Although shown as a single unit, storage 320 may be a combination of fixed or removable storage devices, such as fixed disc drives, removable memory cards or optical storage, network attached storage (NAS), or a storage area-network (SAN).

The storage 320 may include one or more acceptability configurations 338 usable to determine the acceptability of surgical devices, such as the reusable surgical device 232, for subsequent surgical procedures. In various embodiments, the acceptability configurations 338 can include, for example, one or more sets of criteria for determining safety, effectiveness, utility and/or the like based on device-specific profile data such as the profile data 334 for the reusable surgical device 232. In various embodiments, the acceptability configurations 338 can include different configurations for different types of surgical devices, different models of surgical devices, different surgical procedures, and/or the like.

For example, with respect to the reusable surgical device 232, the acceptability configurations 338 can include usage criteria such as a usage threshold associated with a usage limit of the reusable surgical device 232. The usage threshold can conform to a format or type of the usage value in the profile data 334. In an example, if the usage value is increased with each use, the usage threshold may correspond to an upper boundary that is defined in terms of a number of uses, total time in use, etc., as appropriate. In another example, if the usage value is decreased with each use, the usage threshold may correspond to a predetermined lower boundary such as zero. In some cases, the acceptability configurations 338 can include a combination of different thresholds for a variety of different usage values potentially provided in the profile data 334.

By way of further example, with respect to the reusable surgical device 232, the acceptability configurations 338 can include shelf-life criteria such as a shelf-life threshold associated with a shelf-life limit of the reusable surgical device 232. In general, the shelf-life threshold is usable in combination with at least a portion of the profile data 334, such as the manufacturing date, to evaluate shelf life. The shelf-life threshold can be specified in any suitable fashion. For example, the shelf-life threshold can be an amount of time, such that devices manufactured longer ago than that amount of time should not be used (e.g., more than 90 days ago). In another example, the shelf-life threshold can be a specified date, such that devices manufactured prior to the specified date should not be used (e.g., prior to January 1, 2023).

In some cases, some of the acceptability configurations 338 can be specified in terms of a parameter of the profile data 334, with such configuration(s) contemplating an availability of real-time data via other processes of the surgical console 120. For example, the acceptability configurations 338 can include power criteria such as a target power output, where the target power output is a reference to the manufactured power output, for example, from the profile data 334. Evaluation of the target power output may rely on, for example, a measured power output supplied by a calibration process performed prior to, or in parallel with, acceptability assessment. In various cases, the target power output can correspond to the manufactured power output or can be a range that is defined relative to the manufactured power output. The target power output may also be defined in another suitable fashion.

In certain embodiments, the storage 320 may include exception data 340 that serves as a device exclusion list. For example, the exception data 340 can identify surgical devices that should be automatically deemed unacceptable for use in all surgical procedures or certain types of surgical procedures. Such surgical devices can be identified, for example, by manufacturer, manufacturing date, manufacturing lot identifier or the like. The exception data 340 can be based on, for example, product recalls, safety notices, guidelines, or the like.

Memory 318 may include an operating system and/or one or more applications that, when executed by CPU 316, operate the surgical console 120. As shown, the memory 318 includes a device assessment module 336 and an interoperability module 342. In various embodiments, the device assessment module 336, when executed by the CPU 316, monitors utilization of surgical devices. In various embodiments, the interoperability module 342 when executed by the CPU 316 controls one or more parameters of the wireless reader 206, which can improve coupling with various types of reusable surgical devices 232.

More particularly, in certain embodiments, the device assessment module 336 monitors the surgical environment 100 for an indication of an intent to use a surgical device, such as the reusable surgical device 232, in a surgical procedure. When such an indication is received, for example, as a result of the reusable surgical device 232 being placed on the surgical pad 230 or brought in proximity of the external reader surface 250, the device assessment module 336, via the wireless reader 206, reads the profile data 334 and assesses an acceptability of the reusable surgical device 232 according to the acceptability configurations 338 and/or the exception data 340. In general, the device assessment module 336 can establish that the reusable surgical device 232 is acceptable for use in the surgical procedure when there is no basis for deeming it unacceptable (e.g., acceptable by default).

In various embodiments, the device assessment module 336 can establish that the reusable surgical device 232 is unacceptable for use in the surgical procedure based on the acceptability configurations 338. For example, the device assessment module 336 can compare the usage value from the profile data 334 to the usage threshold from the acceptability configurations 338 and determine that the usage threshold has been reached. In another example, the device assessment module 336 can compare manufacturing information from the profile data 334, such as the manufacturing date, to the shelf-life threshold from the acceptability configurations 338, and determine that the shelf-life threshold has been reached. In yet another example, the device assessment module 336 can compare a measured power output of the reusable surgical device 232 to the target power output defined by the acceptability configurations 338 and determine that the target power output is not satisfied, for example, due to a discrepancy between the manufactured power output and the measured power output. In various embodiments, any of the foregoing determinations may result in the reusable surgical device 232 being established as unacceptable for use in the surgical procedure.

In addition, or alternatively, the device assessment module 336 can establish that the reusable surgical device 232 is unacceptable for use in the surgical procedure based on the exception data 340. As mentioned previously, the exception data 340 may include, for example, recall data that is accessible to the device assessment module 336. For example, the device assessment module 336 can compare manufacturing information from the profile data 334, such as the manufacturing lot identifier, to the exception data 340 and determine that at least a portion of the exception data 340 is satisfied. For example, it may be determined that the manufacturing lot identifier matches an identifier for manufacturing lot that has been recalled. In various embodiments, a determination that the reusable surgical device satisfies or matches any of the exception data 340 may result the reusable surgical device 232 being established as unacceptable for use in the surgical procedure.

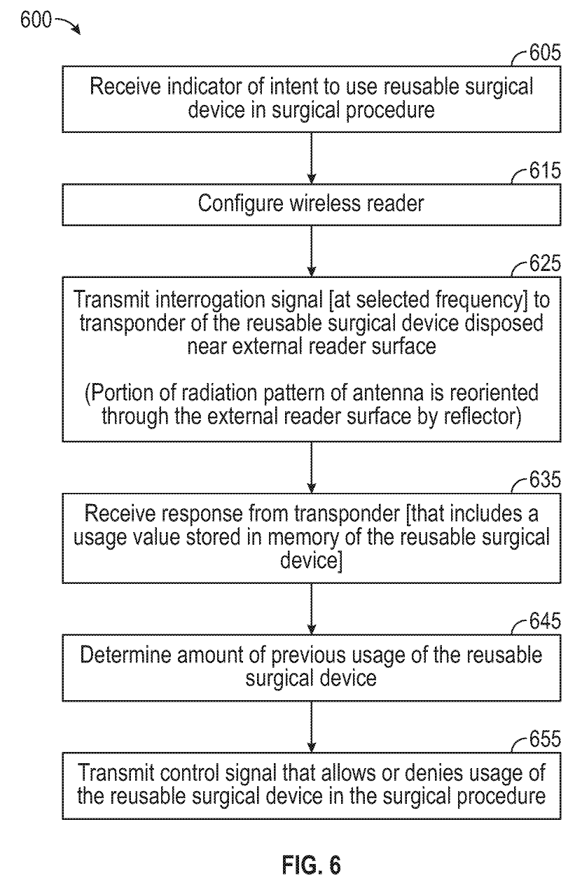

In certain embodiments, the device assessment module 336 allows usage of the reusable surgical device 232 in the surgical procedure in response to a determination that the reusable surgical device 232 is acceptable. Conversely, in a typical embodiment, the device assessment module 336 denies usage of the reusable surgical device 232 in the surgical procedure in response to a determination that the reusable surgical device 232 is unacceptable. In certain embodiments, the device assessment module 336 can update the profile data 334 in conjunction with allowing or denying usage of the reusable surgical device 232. Example operability of the device assessment module 336 to allow or deny usage of the reusable surgical device 232 and update the profile data 334 will be described in greater detail with respect to FIG. 6.

In some embodiments, the interoperability module 342 communicates control signal(s) to the wireless reader 206 that controls one or more parameters of the wireless reader 206. For example, the interoperability module 342 may specify a frequency range or particular frequency at which the wireless reader 206 should operate its antenna 208, a power level, and so forth. In some embodiments, the control signal(s) may be based on identification information for the reusable surgical device 232. In some embodiments, the interoperability module 342 may select the frequency or range based on location information. For example, the surgical console 120 may identify its current location as the United States (U.S.) and the interoperability module 342 selects a frequency range that is known or otherwise approved for use in the U.S., such as 902-928 MHz.

In some embodiments, the interoperability module 342 may select the frequency or range using one or more other criteria, such as being based on historical data of the storage 320 that includes, e.g., a listing of reusable surgical devices 232 that had been previously connected to the surgical console 120, being based on user input to the surgical console 120, and so forth. Further, in some embodiments, the interoperability module 342 may select the frequency or range dynamically, according to a predetermined scheme. For example, the interoperability module 342 may scan for a reusable surgical device 232 by selecting a first frequency or range for a predetermined interval and shifting to subsequent frequencies or ranges when no connection is established with a reusable surgical device 232 during the interval. In some embodiments, the scan may begin responsive to the controller 104 receiving an indication of an intent to use the surgical tool 126 (e.g., a signal from a usage sensor). In other embodiments, the scan may begin responsive to (or concurrently with) other initialization or calibration procedures for the surgical tool 126 that are performed by the controller 104.

Conventionally, operating the antenna 208 of the wireless reader 206 by driving the antenna 208 with frequencies outside the (tuned) bandwidth of the antenna 208 alters the radiation pattern of the antenna 208, such that significantly less energy is able to be wirelessly coupled from the antenna 208 of the wireless reader 206 into the antenna of the transponder 240. In many cases, this results in an inadequate signal strength to establish a connection with the transponder 240. However, according to various embodiments described herein, use of the reflector 220 redirects a portion of the radiation pattern of the antenna 208 to extend through the external reader surface 250, which energy without the reflector 220 would be unavailable to couple into the antenna of the transponder 240. In this way, the wireless reader 206 may connect or have improved connections for different orientations of the transponder 240, and/or may connect or have improved connections with various types of reusable surgical devices 232 having different operational characteristics.

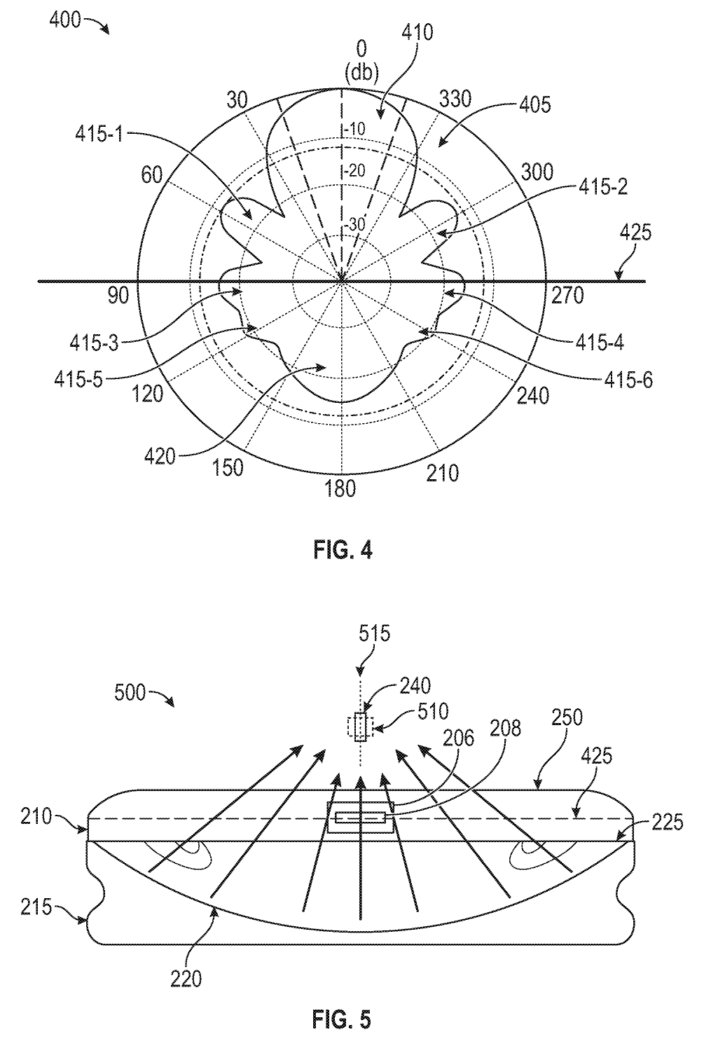

FIG. 4 is a diagram 400 illustrating an exemplary radiation pattern 405 of an antenna according to certain embodiments of the present disclosure. The features illustrated in the diagram 400 may be used in conjunction with other embodiments. For example, the diagram 400 represents a two-dimensional polar coordinate plot that has been shifted 90° to correspond to the orientation of the antenna 208 as it appears in FIG. 5.

The radiation pattern 405 represents a radiation pattern of the antenna 208 within a bandwidth of the antenna 208 and is depicted within a two-dimensional plane. As shown, the radiation pattern 405 defines a plurality of lobes extending from the antenna 208 in different directions. Each of the plurality of lobes represents a local maximum of radiated signal strength from the antenna 208, and adjacent lobes are separated by a respective null where the radiated signal strength is at a local minimum (in some cases, zero).

As shown, the antenna is arranged in a plane 425 extending between 90° and 270°. The plurality of lobes of the radiation pattern 405 includes a main lobe 410 having a highest power or exhibiting the greatest field strength extending at 0°, a back lobe 420 extending opposite the main lobe 410 at 180°, and a plurality of side lobes 415-1, …, 415-6 extending in various directions between 0° and 180°. The plurality of lobes are generally symmetrical about the axis extending between 0° and 180°. As shown, the side lobes 415-1, 415-2 extend in an upward direction from the plane 425, the side lobes 415-3, 415-4 extend in a lateral direction and overlap the plane 425, and the side lobes 415-5, 415-6 extend in a downward direction from the plane 425.

The geometry of the radiation pattern 405 depends on the configuration (e.g., the layout and tuning) of the antenna 208. Referring also to FIG. 5, in implementations without the reflector 220, the antenna 208 may typically be configured such that a maximum amount of the radiation pattern 405 extends through the external reader surface 250 to provide a maximum coupling with the transponder 240. In some embodiments, within a bandwidth of the antenna 208, a majority of the radiation pattern 405 of the antenna 208 extends through the external reader surface 250, the majority of the radiation pattern 405 comprising the main lobe 410. As described herein, a “majority” indicates more than 50% of the energy or field strength radiated by the antenna 208. Using the reflector 220, at least some of the energy radiated in the side lobes 415-3, …, 415-6 and the back lobe 420 may be redirected through the external reader surface 250, improving the coupling with the transponder 240. In some embodiments, the surface of the reflector 220 is contoured such that at least some of the reflected portion of the radiation pattern 405 converges toward one or more foci. As shown, the reflector 220 comprises a parabolic reflector converging at least some of the reflected portion of the radiation pattern 405 toward a focus 510 above the external reader surface 250.

As a result of using the reflector 220, the wireless reader 206 may connect or have improved connections for different orientations of the transponder 240. In some embodiments, using the reflector 220, the wireless reader 206 is configured to connect with the transponder 240 having a perpendicular orientation to the antenna 208. Stated another way, the wireless reader 206 has sufficient coupling to form and/or maintain a connection with the transponder 240 while the plane 425 of the antenna 208 is oriented perpendicular to a plane 515 of the antenna of the transponder 240.

FIG. 6 illustrates a method 600 to monitor usage of a reusable surgical device according to certain embodiments of the present disclosure. The method 600 may be used in conjunction with other embodiments, such as being performed by the device assessment module 336 and/or the interoperability module 342 of the controller 104, in conjunction with the reusable surgical device 232.

The method 600 begins at block 605, where the device assessment module 336 receives an indicator of an intent to use the reusable surgical device 232 in a surgical procedure. The indicator can be any suitable trigger such as, for example, detection of placement of the surgical tool 126 on the surgical pad 230. In some cases, the indicator can be supplied by a user in conjunction with the user placing the surgical tool 126 on the surgical pad 230. Further, in some embodiments, the indicator can be received from another process of the surgical console 120, such as from a calibration process that executes in parallel to the method 600.

At block 615, the interoperability module 342 configures the wireless reader 206. In some embodiments, the interoperability module 342 communicates one or more control signals that control one or more parameters of the wireless reader 206. For example, the interoperability module 342 may specify a frequency range or particular frequency at which the wireless reader 206 should operate its antenna 208, a power level, and so forth. The one or more control signals may be based on location information, historical information, user-input information, and so forth. In some embodiments, the one or more control signals may be selected according to a predetermined scheme (e.g., a scan of frequencies). In some embodiments, the interoperability module 342 transmits a control signal that configures the wireless reader 206 to transmit the interrogation signal at a frequency outside a bandwidth of the antenna 208.

At block 625, the wireless reader 206 transmits an interrogation signal to a transponder 240 of the reusable surgical device 232, which is disposed near an external reader surface 250 of the wireless reader 206. A portion of a radiation pattern of the antenna is reoriented through the external reader surface 250 by a reflector 220. In some embodiments, the interrogation signal is transmitted at a frequency selected at block 615.

At block 635, the wireless reader 206 receives a response from the transponder. In some embodiments, the response includes a usage value stored in a memory of the reusable surgical device 232. The response may include other types of information in the profile data 334, such as a device identifier, a manufacturing date, a manufacturing lot identifier, a manufactured power output, and so forth.

At block 645, the device assessment module 336 determines an amount of previous usage of the reusable surgical device 232. At block 655, the device assessment module 336 automatically transmits a control signal that allows or denies usage of the reusable surgical device 232 in the surgical procedure. In some embodiments, the control signal is based on the amount of previous usage of the reusable surgical device 232 and optionally one or more assessment criteria.

When the control signal denies usage of the reusable surgical device 232, the control signal may cause one or more actions that prevent usage of the reusable surgical device 232, such as advising or informing the user of the surgical console 120, aborting other processes of the surgical console 120 (e.g., a calibration process relative to the reusable surgical device 232), notifying one or more designated users inside or outside the surgical environment, halting a current workflow, initiating a different exceptional workflow, halting delivery of operational power to the reusable surgical device 232 (e.g., electrical power, mechanical power, pressurized fluid, vacuum, etc.) and so forth.

In some embodiments, the device assessment module 336 optionally updates the profile data 334 in the transponder 240 based on operation of the method 600. For example, when usage of the reusable surgical device 232 is allowed, the usage value can be updated in the memory of the reusable surgical device 232. In another example, when usage of the reusable surgical device 232 is denied, the usage value can be set to the usage threshold so that no further uses will be allowed. In another example, the usage value can be set to a predetermined value (e.g., a negative number) to indicate a safety issue with the reusable surgical device 232. The method 600 ends following completion of the block 655.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. An apparatus to monitor usage of a reusable surgical device, the apparatus comprising:
a housing defining an external reader surface;
a wireless reader comprising an antenna disposed within the housing, wherein within a bandwidth of the antenna, a majority of a radiation pattern of the antenna extends through the external reader surface; and
a reflector disposed near the antenna opposite the external reader surface, wherein the reflector is configured to reorient a portion of the radiation pattern as a reflected portion that extends through the external reader surface.

2. The apparatus of claim 1, wherein the reflector comprises a parabolic reflector configured to converge at least some of the reflected portion toward a focus above the external reader surface.

3. The apparatus of claim 1, wherein the majority of the radiation pattern comprises a main lobe of the radiation pattern, and where the portion of the radiation pattern comprises a back lobe of the radiation pattern.

4. The apparatus of claim 1, wherein the wireless reader is configured to connect with a transponder of the reusable surgical device having a perpendicular orientation to the antenna.

5. The apparatus of claim 1, wherein the reflector is disposed within the housing.

6. The apparatus of claim 1, wherein the housing is integrated into a surgical tray.

7. The apparatus of claim 1, wherein the wireless reader is connected to a surgical console and is configured to:
receive an indicator of an intent to use the reusable surgical device in a surgical procedure;
access a usage value stored in a memory of the reusable surgical device; and
transmit the usage value to the surgical console.

8. The apparatus of claim 7, wherein the wireless reader is further configured to:
receive a signal from the surgical console indicating whether usage of the reusable surgical device in the surgical procedure is allowed; and
update the usage value in the memory of the reusable surgical device.

9. The apparatus of claim 7, wherein the wireless reader is further configured to:
transmit, responsive to receiving the indicator, an interrogation signal to a transponder of the reusable surgical device.

10. The apparatus of claim 9, wherein the wireless reader is further configured to:
receive a control signal from the surgical console; and
transmit, responsive to the control signal, the interrogation signal at a frequency outside the bandwidth of the antenna.

11. The apparatus of claim 10, wherein the control signal is based on identification information for the reusable surgical device.

12. A method to monitor usage of a reusable surgical device, the method comprising:
transmitting, using an antenna of a wireless reader, an interrogation signal to a transponder of the reusable surgical device disposed near an external reader surface, wherein a portion of a radiation pattern of the antenna is reoriented through the external reader surface by a reflector that is disposed near the antenna opposite the external reader surface;
receiving, at the wireless reader, a response from the transponder;
determining, by a surgical console connected with the wireless reader, an amount of previous usage of the reusable surgical device that is based at least partly on the response; and
transmitting, by the surgical console, a first control signal that allows or denies usage of the reusable surgical device in a surgical procedure, the first control signal based at least partly on the amount of previous usage.

13. The method of claim 12, wherein the reflector comprises a parabolic reflector configured to converge at least some of the portion of the radiation pattern of the antenna toward a focus above the external reader surface.

14. The method of claim 12, further comprising:
receiving an indicator of an intent to use the reusable surgical device in the surgical procedure,
wherein transmitting the interrogation signal is responsive to receiving the indicator.

15. The method of claim 12, wherein:
the response includes a usage value stored in a memory of the reusable surgical device; and
the usage value is transmitted to the surgical console.

16. The method of claim 15, further comprising:
transmitting, by the surgical console, a second control signal to the reusable surgical device, causing the usage value to be updated in the memory of the reusable surgical device.

17. The method of claim 16, wherein the usage value is updated to a predetermined value that indicates a safety issue with the reusable surgical device.

18. The method of claim 12, further comprising:
transmitting, by the surgical console, a second control signal to configure the wireless reader to transmit the interrogation signal at a frequency outside a bandwidth of the antenna.

19. The method of claim 12, wherein transmitting the first control signal causes a calibration process relative to the reusable surgical device to abort.

20. The method of claim 12, wherein transmitting the first control signal causes delivery of operational power to the reusable surgical device to be halted.
